(19) United States
(12) Patent Application Publication
(10) Pub. No.: US 2022/0305708 A1
(43) Pub. Date: Sep. 29, 2022

(54) Cement Spacer Mold System

(71) Applicant: Brent Austin McNeely, Breinigsville, PA (US)

(72) Inventor: Brent Austin McNeely, Breinigsville, PA (US)

(21) Appl. No.: 17/842,238

(22) Filed: Jun. 16, 2022

Related U.S. Application Data
(63) Continuation-in-part of application No. 17/307,890, filed on May 4, 2021.
(60) Provisional application No. 63/020,158, filed on May 5, 2020.

(51) Int. Cl. B29C 45/26; B29C 45/64; A61B 17/84

(52) U.S. Cl. CPC B29C 45/261; B29C 45/64; A61B 17/846; B29K 2033/12

(57) ABSTRACT

A medical nail mold suited for casting fusion nails is presented. The medical nail mold contains a base with a base cavity, a tube with a cavity, a locking mechanism to hold together the base and the tube, and a core holder. The base and the tube are connected to align the base cavity with the tube cavity and are locked in place by the locking mechanism. The core holder is aligned with the cavity opposite the base to stabilize a core during use.

### FIELD OF THE INVENTION

The present invention relates to cement spacer mold within the medical field. The present invention provides a bone cement spacer mold system to create a bone cement mold for patients in need, such as patients under one-stage or two-stage revision arthroplasty/arthrodesis.

### BACKGROUND OF THE INVENTION

Within the medical fields, bone cement spacer molding can be an issue when it comes to specifics qualities that are needed for patients. There are few known bone cement spacer molds in which exist but seemingly each specific mold can only cover a narrow form or placement of the bone spacer that is created from the molds. One example is the difficulty with centering a Kirschner wire within the bone cement. Many mold spacers are designed horizontally which can cause the bone cement to be uneven which causes uneven centering of the Kirschner wire. Another issue that arises from many of existing bone cement spacer mold is that the accessibility of different sized bone cement created from the molds are limited to the mold itself. Many existing bone cement spacer molds are unable to create different lengths of bone cement as each patient are different from one another and desire different length of bone cement to satisfy their needs. Further, some bone cement spacer molds can lead to flashing and creation of bubbles in the cement if the casting process is not executed perfectly, leading to imperfect or unusable bone spacers.

An objective of the present invention is to provide the user a way of making bone cement molds. The bone cement spacer mold is able to create a variety length of bone cement to satisfy the user's needs for such applications in revising Tibiotalocalcaneal arthrodesis (TTC) and/or other long bone/screws removals and revisions. Also note that the present invention allows the Kirschner wire or core to be centered along the body of the mold as to other bone cement spacer molds. Furthermore, the present invention provides a mean of long-term and short-term use of the device as there are reusable molds and disposable molds.

### SUMMARY OF THE INVENTION

The present invention is a medical nail mold suited for casting ankle fusion nails. The medical nail mold comprises a base, a tube, a cap, a core holder, and a locking mechanism. The base and the tube connect and lock into place with the locking mechanism. The cap is positioned on one end of the tube opposite the base. The core holder attached to the cap and is configured to hold a core stationary within the tube. In casting a medical nail, bone cement is injected into a cavity in the tube and a core is inserted into the cement via the core holder.

### BRIEF DESCRIPTION OF THE DRAWINGS

FIG. 1 is a top perspective view of the present invention in accordance with at least one embodiment.

FIG. 2 is a bottom perspective view of the present invention in accordance with at least one embodiment.

FIG. 3 is a front elevation view of the present invention in accordance with at least one embodiment.

FIG. 4 is a top plan view of the present invention in accordance with at least one embodiment.

FIG. 5 is a top perspective view of the base of the present invention in accordance with at least one embodiment.

FIG. 6 is a top perspective view of the tube of the present invention in accordance with at least one embodiment.

FIG. 7 is a bottom perspective view of the tube of the present invention in accordance with at least one embodiment.

FIG. 8 is a top perspective view of the core holder of the present invention with an attached core in accordance with at least one embodiment.

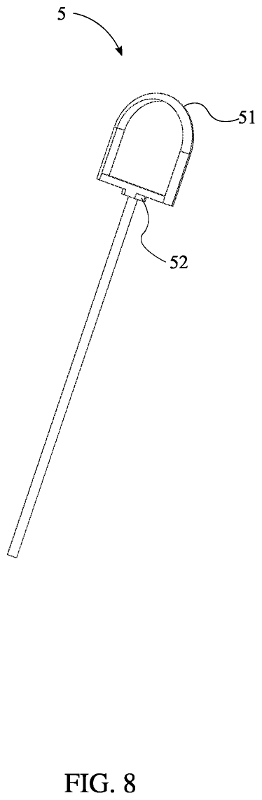

FIG. 9 is an exploded view of the present invention in accordance with at least one embodiment.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

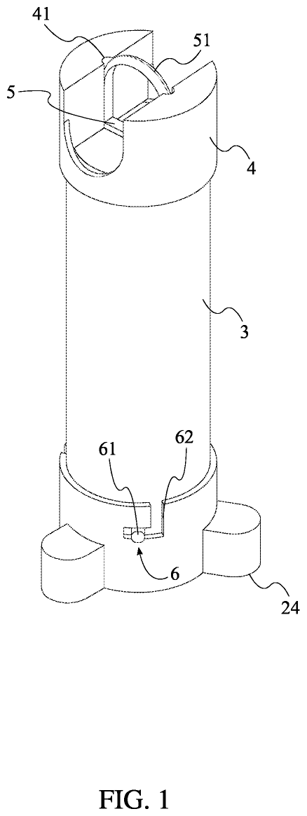

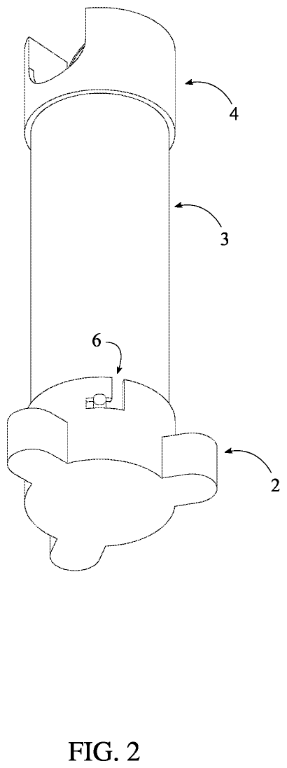

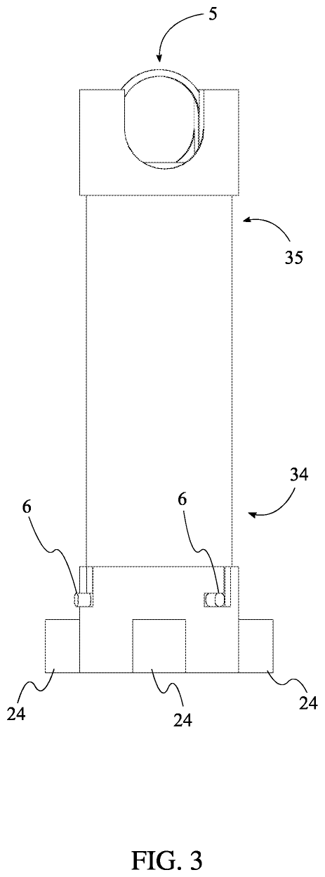

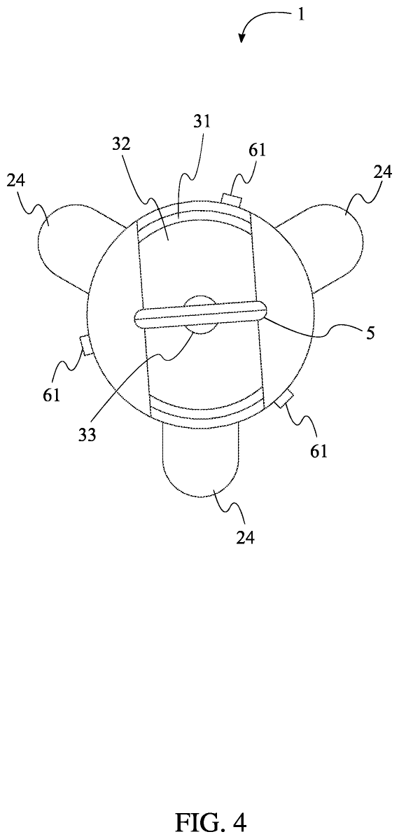

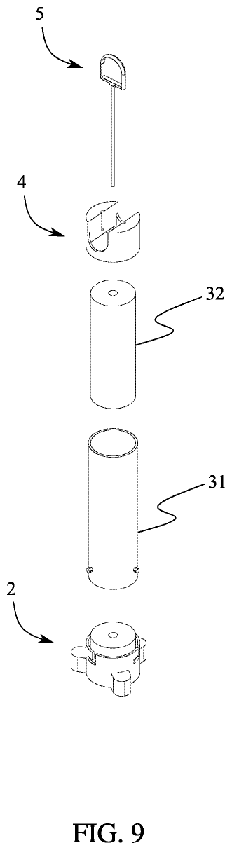

In reference to FIGS. 1-4 and 9, the present invention is a medical nail mold 1 suited for casting a cast medical nail. The medical nail mold 1 comprises a base 2, a tube 3, a cap 4, a core holder 5, and a locking mechanism 6. As shown in FIG. 1-3, the tube 3 is aligned with the base 2 and connected by the locking mechanism 6. The cap 4 attaches to the tube 3 opposite the base 2 and the core holder 5 is inserted into the tube 3 and held in place by the cap 4. The components of the medical nail mold 1 may be connected in a particular order in accordance with a process for casting a medical nail, as discussed later in this description.

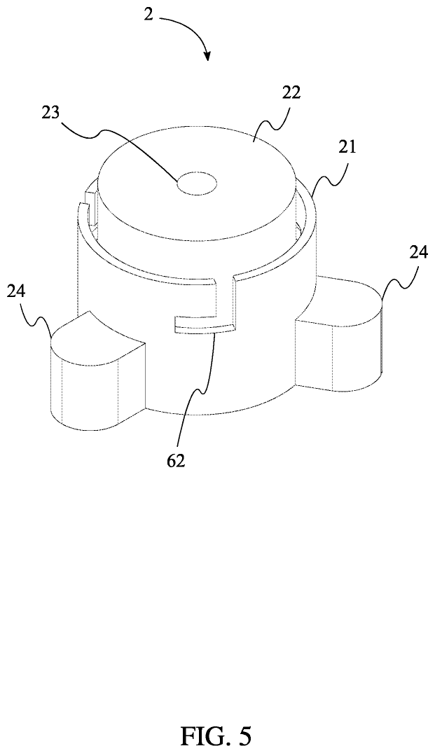

The base 2 of the present invention further comprises an outer ring 21, a mold base 22, a base cavity 23, and a plurality of feet 24. As shown in FIG. 5, in the preferred embodiment, the mold base 22 is a cylindrical element concentrically positioned inside the outer ring 21, with space between the outer ring 21 and the mold base 22. The base cavity 23 is a depression in the center of the mold base 22. The base cavity 23 is preferably semi-spherical in shape, though in some embodiments, the base cavity 23 may be rectangular, cylindrical, conical, or any other shape desired for the tip end of a medical nail. The plurality of feet 24 extend outward from the cylindrical structure of the base 2, increasing the stability of the base 2. The mold base 22 is made out of a silicone material or any other suitable casting mold material. In the preferred embodiment of the present invention, the outer ring 21 and the plurality of feet 24 of the base 2 are made out of a rigid material, such as, but not limited to stainless steel, aluminum, polymer, or other suitable material.

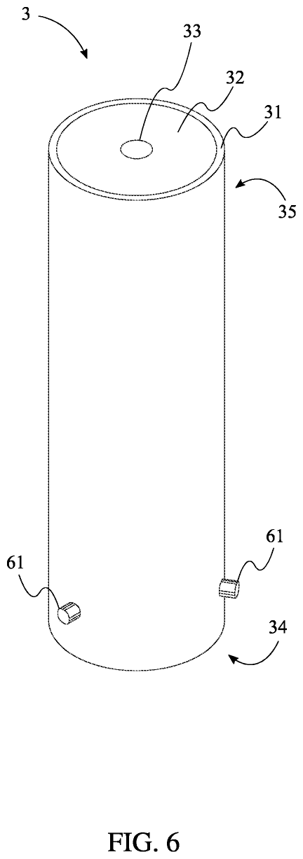

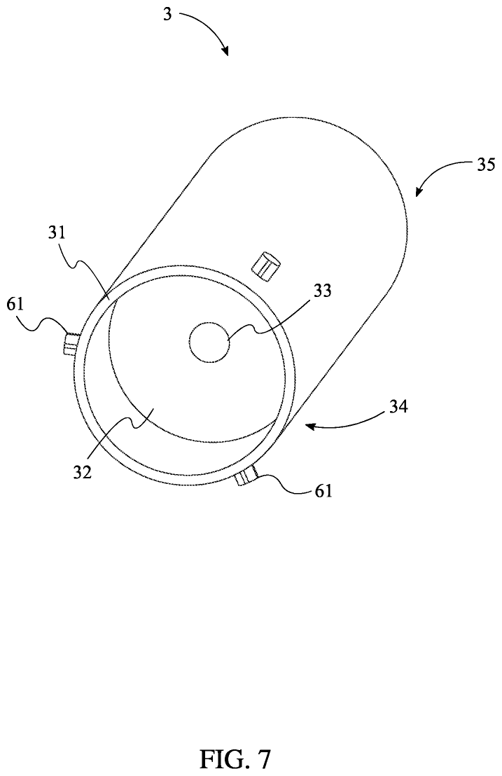

Regarding FIG. 6-7, the tube 3 of the present invention further comprises a sleeve 31, a mold body 32, a cavity 33, a bottom end 34, and a top end 35. The mold body 32 is a cylindrical piece of material concentrically positioned within the sleeve 31. The sleeve 31 is a thin cylindrical casing surrounding and in contact with the mold body 32. The cavity 33 is a cylindrical opening in the center of the mold body 32, passing entirely through the mold body 32 from the top end 35 to the bottom end 34. Similar to the base 2, the mold body 32 is made out of a silicone material or any other suitable casting mold material and the sleeve 31 is made out of a rigid material, such as, but not limited to stainless steel, aluminum, polymer, or other suitable material. When the components of the medical nail mold 1 are put together, the bottom end 34 of the tube 3 connects with the base 2. In the preferred embodiment, the mold body 32 and the mold base 22 have the same circular dimensions and are placed against each other with the cavity 33 and the base cavity 23 also having the same circular dimensions and being aligned at their point of contact. The sleeve 31 may extend further than the mold body 32 on the bottom end 34 of the tube 3, allowing the sleeve 31 to slidingly engage with the space between the outer ring 21 and the mold base 22 when the base 2 is connected to the tube 3.

Connection of the base 2 and the tube 3 may be facilitated by the locking mechanism 6 of the present invention. The locking mechanism 6 securely holds together the base 2 and the tube 3 of the medical nail mold 1 during use. In the preferred embodiment, as shown in FIG. 1-3, the locking mechanism 6 comprises a plurality of prongs 61 and a plurality of channels 62. The number of channels in the plurality of channels 62 matches the number of prongs in the plurality of prongs 61 with each prong of the plurality of prongs 61 being configured to slide into a different channel of the plurality of channels 62. In the preferred embodiment, the plurality of prongs 61 is attached to the tube 3 and the plurality of channels 62 is carved out of the outer ring 21 of the base 2, though this orientation may also be inverted. Preferably, each of the plurality of channels 62 has an L-shape, allowing the plurality of prongs 61 to be inserted into the plurality of channels 62 and twisted to prevent the base 2 and the tube 3 from being pulled apart from one another. While described here in the preferred embodiment, the locking mechanism 6 may take another form to hold together the base 2 and the tube 3.

The cap 4 of the present invention connects to the tube 3 at the top end 35. The cap 4 partially covers the top end 35 of the tube 3, while still allowing access to the cavity 33 at the top end 35 of the tube 3. The cap comprises at least one holder groove 41 to keep in place the core holder 5 of the present invention during use. In the preferred embodiment, the at least one holder groove 41 of the cap 4 is a pair of semi-circular depressions in the cap 4 that match the shape of the core holder 5, keeping it from tilting or rotating during use.

The core holder 5 of the present invention connects to a core to be used as the center structural component around which a cement spacer is formed. The core holder 5 comprises a handle 51 and a core attachment 52. In the preferred embodiment, the handle 51 of the core holder 5 is a ring shape to be easily grabbed and moved by a user. The shape of the handle 51 matches the shape of the at least one holder groove 41 to allow the handle to lock into place with the cap 4 as not to tilt, twist, or otherwise move the core during use. The core attachment 52 is a mechanism attached to the handle 51 that engages with the core. In some embodiments, this core attachment 52 may simply be a chunk of material with a hole for inserting one end of the core.

In use, the medical nail mold 1 is set up to cast a medical nail through the following steps. First, cement is injected into the cavity 33 at the top end 35 of the tube 3 until the cavity 33 is completely filled with cement and the cement comes out the cavity 33 at the bottom end 34 of the tube 3. The bottom end 34 of the tube 3 is then placed against the base 2, aligning the mold body 32 with the mold base 22 and aligning the cavity 33 with the base cavity 23. In doing this, the excess cement from the cavity 33 enters into and fills the base cavity 23. The base 2 and tube 3 may then be secured together using the locking mechanism 6. In the preferred embodiment, this involves inserting the plurality of prongs 61 into the plurality of channels 62 and twisting the tube 3 and the base 2 is opposite directions to lock them together. Next, the core holder 5 is attached to the cap 4, which is connected to the top end 34 of the tube 3. In attaching the core holder to the cap, the core is connected to the core attachment 52, then the core is inserted into the cavity 33 from the top end 35 of the tube 3 and the core and core holder 5 are locked in place by engaging the handle 51 of the core holder 5 with the at least one holder groove 41 of the cap 4.

This design is an improvement over similar systems of casting bone spacers because the connection of the tube 3 and the base 2 after cement has been poured through the tube 3 reduces flashing and helps eliminate bubbles from forming in the cement as it cures. In addition to limiting flashing overall, the present invention minimizes the risk of flashing to the small area of contact between the tube 3 and the base 2. By eliminating flashing/bubbles in the present medical nail mold 1, sufficiently strong medical nails can be made on a more reliable basis with less skill and precision by the user and the completed medical nails can be more easily removed from the invention.

The length of the tube 3 and the size of the cavity 33 and base cavity 23 may differ depending on the desired size of the bone spacer. For example, the length of the tube 3 may be shortened or lengthened to account for shorter or longer bone spacers and the width of the cavity 33 and the base cavity 23 may be adjusted to make thinner or thicker bone spacers. In some embodiments, the tube 3 may be interchangeable with other tubes 3 that have various lengths and cavity widths. Further, in some embodiments, the mold body 32 and/or the mold base 22 may be removable from the tube 3 and the base 2, allowing the mold material or the dimensions of the cavity 33 and base cavity 23 to be adjusted without using an entirely separate medical nail mold 1.

Although the invention has been explained in relation to its preferred embodiment, it is to be understood that many other possible modifications and variations can be made without departing from the spirit and scope of the invention as hereinafter claimed.

What is claimed is:

1. A medical nail mold comprising:
a base;
a tube;
a locking mechanism;
a core holder;
the base having a base cavity passing partially into the base, centered on the base;
the tube having a top end, a bottom end, and a cavity passing entirely through the tube from the top end to the bottom end, centered on the tube;
the bottom end of the tube being connected to the base by the locking mechanism, wherein the tube and the base are separable by engaging the locking mechanism;
the cavity at the bottom end of the tube being connected to the base cavity; and
the core holder being connected to the top end of the tube.

2. The medical nail mold of claim 1, further comprising:
the locking mechanism further comprising a plurality of prongs and a plurality of channels;
the plurality of prongs and the plurality of channels being attached to the base and the tube;
each of the plurality of prongs being configured to engage with one of the plurality of channels, wherein engaging the plurality of prongs with the plurality of channels prevents separation of the tube and the base.

3. The medical nail mold of claim 1, wherein the base cavity is semispherical in shape.

4. The medical nail mold of claim 1, further comprising:
a cap;
the cap attached to the top end of the tube;
the cap comprising at least one holder groove; and
the core holder being connected to the cap by the at least one holder groove.

5. The medical nail mold of claim 4, further comprising:
the core holder further comprising a handle and a core attachment;
the handle being connected to the at least one holder groove of the core holder, wherein the connection between the handle and the holder groove restricts angular movement of the core holder; and
the core attachment being configured to accept and secure a medical nail core.

6. The medical nail mold of claim 1, further comprising:
the base comprising a mold base, an outer ring, and a plurality of legs;
the base cavity passing partially into the mold base;
the mold base concentrically positioned inside the outer ring; and
the plurality of legs radially extending from the base.

7. The medical nail mold of claim 6, further comprising:
the mold base being made of a silicone material; and
the outer ring and the plurality of legs being made of a rigid material.

8. The medical nail mold of claim 1, further comprising:
the tube comprising a sleeve and a mold body;
the cavity passing entirely through the mold body; and
the mold body being concentrically positioned inside the sleeve.

9. The medical nail mold of claim 8, further comprising:
the mold body being made of a silicone material; and
the sleeve being made of a rigid material.

10. A medical nail mold, comprising:
a base;
a tube;
a cap;
a locking mechanism;
a core holder;
the base having a mold base, an outer ring, a plurality of legs, and a base cavity; the base cavity passing partially into the mold base, centered on the base;
the tube having a top end, a bottom end, a sleeve, a mold body and a cavity;
the cavity passing entirely through the mold body from the top end to the bottom end, centered on the tube;
the cap having at least one holder groove;
the core holder having a handle and a core attachment;
the bottom end of the tube being connected to the base by the locking mechanism, wherein the tube and the base are separable by engaging the locking mechanism;
the cavity at the bottom end of the tube being connected to the base cavity; and
the core holder being connected to the top end of the tube;
the cap being attached to the top end of the tube;
the handle being connected to the at least one holder groove of the core holder, wherein the connection between the handle and the holder groove restricts angular movement of the core holder; and
the core attachment being configured to accept and secure a medical nail core.

11. The medical nail mold of claim 10, further comprising:
the locking mechanism further comprising a plurality of prongs and a plurality of channels;
the plurality of prongs and the plurality of channels being attached to the base and the tube;
each of the plurality of prongs being configured to engage with one of the plurality of channels, wherein engaging the plurality of prongs with the plurality of channels prevents separation of the tube and the base.

12. The medical nail mold of claim 10, wherein the base cavity is semispherical in shape.

13. The medical nail mold of claim 10, further comprising:
the mold body and the mold base being made of a silicone material; and
the sleeve, the outer ring, and the plurality of legs being made of a rigid material.
